(19) United States
(12) Patent Application Publication
(10) Pub. No.: US 2019/0079094 A1
(43) Pub. Date: Mar. 14, 2019

(54) SUBCELLULAR LOCALIZATION OF TARGET ANALYTES

(71) Applicant: Beckman Coulter, Inc., Brea, CA (US)

(72) Inventors: George C. Brittain, Miami, FL (US); Sergei Gulnik, Miami, FL (US)

(21) Appl. No.: 16/084,513

(22) PCT Filed: Mar. 16, 2017
(86) PCT No.: PCT/US2017/022802

§ 371 Date: Sep. 12, 2018

Related U.S. Application Data
(60) Provisional application No. 62/310,595, filed on Mar. 18, 2016.

(51) Int. Cl. G01N 33/58; G01N 15/14

(52) U.S. Cl. CPC G01N 33/58; G01N 2015/1486; G01N 2458/10; G01N 15/1459

(57) ABSTRACT

The present invention provides methods of determining and quantifying the subcellular localization of an analyte within a sample of cells by using at least two permeabilizing reagents.

### CROSS-REFERENCES TO RELATED APPLICATIONS

This application claims priority to U.S. Provisional Application No. 62/310,595, filed on Mar. 18, 2016, the contents of which are incorporated by reference herewith in their entirety.

### FIELD OF THE INVENTION

This invention relates to methods, articles and compositions for the subcellular detection and analysis of target analytes in cell samples.

### BACKGROUND OF THE INVENTION

The analysis of intracellular markers by flow cytometry, relies on the measurement of the absolute signal emitted by each stain or fluorescent marker present within each cell. These data do not confer the subcellular localization of such signals, and leave the user to infer the localization by existing knowledge of the stain or target molecule, if available. For example, when analyzing the activation of activatable proteins such as transcription factors, the only existing method by traditional flow cytometry is to analyze the levels of their phosphorylation or other modification and assume that this information correlates with eventual nuclear localization.

An important factor when analyzing any of these molecules is whether or not they are actually present or translocated into the nucleus. In some cases, such as with members of the Signal Transducers and Activators of Transcription (STAT) family, this information may be reasonably accurate, since the STATs immediately translocate into the nucleus once phosphorylated. However, this is not always the case because cell signaling is often quite complex, and most proteins have a circuitous set of events required prior to translocation into the nucleus. Additional activation steps may also be required to initiate transcriptional modification once within the nucleus.

Further, any method that relies solely on modification states, without information about subcellular localization is hampered by a variety of issues, including: 1) The necessity for useful antibodies to such modifications; 2) The fact that there are numerous different types of modifications to each and every protein/molecule that all require their own antibodies that may not exist (e.g., phosphorylation, carbamylation, methylation, acetylation, sulfonation, nitrosylation, ubiquitination, etc.); 3) The fact that most modifications have not actually been identified for most proteins/molecules; 4) The ephemeral nature of modification states, which does not necessarily correlate directly with the subcellular localization of the proteins over time or with the protein expression levels themselves (i.e., protein that is no longer modified may still be present and functioning within the target compartment); 5) The compatibility of the permeabilization kit that is utilized for assessing such modifications; 6) And, the requirement for either the presence of the modification on the surface of the molecule being analyzed or the biochemical exposure of such modification in order to enable access of the antibody to the modification for staining. Indeed, although phosphorylation correlates perfectly with the induction of nuclear translocation for the STAT family, the latter issue renders the assessment of STAT phosphorylation impossible by all but the most harsh fixation/permeabilization kits on the market, which typically have issues with detecting other proteins due to their harshness.

Imaging flow cytometry, using low- to moderate-resolution microscopic images of cells as they pass through the cytometer, has been used for visual assessment of the subcellular localization of proteins. Alternatively, cells have been purified and then analyzed either by traditional microscopy, western blotting of protein lysates following biochemical cell subfractionation, or other molecular biochemical methods.

These prior-art methods all have disadvantages. Imaging flow cytometry requires expensive instrumentation. It is also primarily qualitative, and since it takes two-dimensional images of three-dimensional cells, may not effectively distinguish the cytoplasmic vs. nuclear localization of perinuclear proteins or proteins within compartments that are located in front of or behind the nucleus in the image. Similarly, traditional microscopy works well, though is mostly qualitative and has difficulty resolving the three dimensional localization of perinuclear proteins. More advanced microscopic techniques, such as confocal microscopy, mostly resolve this issue by taking numerous image slices of the cell, and then allowing them to be reconstructed into a three dimensional image; however, these microscopes are much more expensive than an image cytometer, and they work best with cells that are adherent to microscope slides. In addition, even with the most advanced microscopes, it is still difficult to discern whether perinuclear membrane-bound proteins are located inside or outside of the nuclear membrane.

The primary disadvantage of molecular biochemical techniques is the time and care required to process and prepare the protein extracts for analysis, which can take days for most techniques, including western blotting. In addition, a major disadvantage that is common to both microscopy and molecular biochemical techniques when they are used for analyzing complex samples, such as whole blood, is that it is necessary to first purify the target cell population and then rest, culture, and possibly expand the cells for days to weeks prior to further experimentation and analyses.

The present invention addresses these and other disadvantages of prior-art methods for detecting subcellular localization of target analytes, such as activatable proteins.

### BRIEF SUMMARY OF THE INVENTION

The present invention provides methods for quantifying an analyte within a sample of cells. The method comprises treating a first aliquot of the cells with a first permeabilizing reagent that permeabilizes the cytoplasmic membrane but does not permeabilize the nuclear membrane; treating a second aliquot of the cells with a second permeabilizing reagent that permeabilizes both the cytoplasmic membrane and the nuclear membrane; washing the first and second aliquots with washing buffer, such as PBS with or without BSA or FBS; staining the first aliquot and the second aliquot with a labeled reagent capable of specifically binding to the analyte; measuring a first signal from the labeled reagent in a cell of the first aliquot and a second signal from the labeled reagent in a cell of the second aliquot; and, comparing the first signal to the second signal to determine the distribution of the analyte. The analyte can be an activatable protein or a protein differentially expressed or activated in diseased or aberrant cells, including but not limited to transcription factors or regulators, such as members of the NF-κB, Rel, STAT, TRAF, FoxP, FoxO, Catenin, CREB, ATF, steroid receptor, HOX, TFII, Histone Acetyltransferase, Histone Deacetylase, SP-1, Activator Protein, C/EBP, E4BP, NFIL, p53, Heat Shock Factor, Jun, Fos, Myc, Oct, NF-I, or NFAT families; kinases, such as members of the ERK, AKT, GSK, MAPK, MAP2K, MAP3K, MAP4K, MAP5K, MAP6K, MAP7K, MAP8K, PI3K, CaM, PKA, PKC, PKG, CDK, CLK, TK, TKL, CK1, CK2, ATM, ATR, GPCR, or receptor tyrosine kinase families; phosphatases, such as members of the MKP, SHP, calcineurin, PP1, PP2, PPM, PTP, CDC, CDC14, CDKN3, PTEN, SSH, DUSP, protein serine/threonine phosphatase, PPP1-6, alkaline phosphatase, CTDP1, CTDSP1, CTDSP2, CTDSPL, DULLARD, EPM2A, ILKAP, MDSP, PGAM5, PHLPP1-2, PPEF1-2, PPTC7, PTPMT1, SSU72, UBLCP1, myotubularins, receptor tyrosine phosphatase, nonreceptor-type PTPs, VH-1-like or DSP, PRL, or atypical DSP families; DNA and/or RNA-binding and modifying proteins, such as members of the histone, single-stranded DNA binding protein, double-stranded DNA binding protein, zinc-finger protein, bZIP protein, HMG-box protein, leucine-zipper protein, nuclease, polymerase, ligase, helicase, transcription factor, co-activator, co-repressor, scaffold protein, endonuclease, exonuclease, recombinase, telomerase, polyadenylase, RNA splicing enzyme, and ribosome families; nuclear import and export receptors; regulators of apoptosis or survival, including members of the BCL2 family and the variety of checkpoint proteins; and ligases of the ubiquitin and ubiquitin-like protein families and their respective deconjugating enzymes, such as members of the deuquitinase, deSUMOylase, delSGylase, USP, and cysteine protease families. The analyte may also be proteins typically constitutively present in one compartment or another, including but not limited to structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, and ion channels. The analyte may also be DNA, chromosomes, oligonucleotides, polynucleotides, RNA, mRNA, tRNA, rRNA, microRNA, peptides, polypeptides, proteins, lipids, ions, sugars (such as monosaccharides, oligosaccharides, or polysaccharides), lipoproteins, glycoproteins, glycolipids, or fragments thereof.

The method can include measuring the signals on a cell-by-cell basis, such as by flow cytometry, imaging flow cytometry, or mass cytometry. Samples may also be analyzed using other cytometric methods, such as microscopy.

The method can also include treating a third aliquot of the cells with a third permeabilizing reagent that permeabilizes the cytoplasmic and one or more organelle membranes, with or without permeabilizing the nucleus.

The first permeabilizing reagent may include between 0.001 and 0.25% Digitonin. For example, the first reagent may include about 0.01-0.15% Digitonin, about 1-100 mM MES with a pH of 4.5-6.5, 0-274 mM NaCl and 0-5.2 mM KCl.

The second permeabilizing reagent may include one of >0.01% Digitonin or >0.0125% TX-100. In some embodiments, the second reagent may include one of about 0.025-0.5% Digitonin or about 0.0125-0.25% Triton X-100. The second reagent may also include about 1-100 mM MES with a pH of 4.5-6.5, 0-274 mM NaCl and 0-5.2 mM KCl.

In some embodiments, the methods include a step of fixing the cells with a fixative, such as 1-10% paraformaldehyde.

The target cells may consist of polymorphonuclear cells (e.g., granulocytes), where the first permeabilizing reagent could include one of a mixture of about 0.01-0.15% Digitonin and about 0.0125-0.25% TX-100 to permeabilize the cytoplasmic membrane, and the second reagent a mixture of about 0.01-0.15% Digitonin and >0.0125% Tween 20 to permeabilize the cytoplasmic+nuclear membranes, or >0.05% Tween 20 to permeabilize the cytoplasmic+mitochondrial membranes.

The method may include the step of staining the first aliquot and the second aliquot with a labeled reagent capable of specifically binding to a surface marker of the cells.

The invention also provides kits for carrying out the methods of the invention. A kit may comprise a first permeabilizing reagent that permeabilizes the cytoplasmic membrane of the cells but not the nuclear membrane; and a second permeabilizing reagent that permeabilizes both the cytoplasmic and nuclear membranes of the cells. The first permeabilizing reagent may include one of about 0.01-0.15% Digitonin or a mixture of about 0.01-0.15% Digitonin and about 0.0125-0.25% TX-100. The second permeabilizing reagent may include one of about 0.025-0.5% Digitonin, 0.0125-0.25% TX-100, 0.01-0.15% Digitonin and >0.0125% Tween20, or >0.05% Tween 20. The kit may further comprise a fixative.

### BRIEF DESCRIPTION OF THE DRAWINGS

FIG. 1 is a workflow for lysing whole blood using the methods of the invention.

FIGS. 2A and 2B. Digitonin and TX-100 Titrations to Determine the Optimal Concentrations for Cytoplasmic vs. Nuclear Membrane Permeabilization. A) The cytoplasmic membrane in whole blood is fully permeabilized around 0.031% Digitonin, while the nucleus is also permeabilized around 0.5% Digitonin or 0.125% TX-100. B) The cytoplasmic membrane in PBMCs is fully permeabilized around 0.0016% Digitonin, while the nucleus is also permeabilized around 0.05% Digitonin or 0.025% TX-100. In this figure, the reduction in Calcein signal is indicative of permeabilization of the plasma membrane, while the peaked HDAC1 staining indicates complete nuclear permeabilization. The ledge that forms with HDAC1 prior to complete lysis is due to lysis of the endoplasmic reticulum, which also contains HDAC1.

FIG. 3. Titrations of Digitonin and TX-100 to Determine the Optimal Concentrations to Permeabilize the Cytoplasm vs. Nucleus of MCF-7 Cells. A) Digitonin permeabilized the cytoplasm by 0.031% and the nucleus by 0.25%. B) TX-100 permeabilized the cytoplasm by 0.0156% and the nucleus by 0.125%. In this figure, permeabilization of the cytoplasmic membrane is indicated by HSP60 staining, while permeabilization of the nuclear membrane is indicated by HDAC1 staining.

FIG. 4. Modified Protocol to Assess the Permeabilization of the Plasma Membrane with MCF-7 Cells. The cells were preloaded with CytoCalcein Violet, and cytoplasmic membrane permeabilization is indicated by the loss of this signal. In this experiment, 0.025% Digitonin or TX-100 permeabilized only the cytoplasm, while 0.25% of either fully permeabilized the both cytoplasmic and nuclear membranes.

FIGS. 5A and 5B. Cytoplasmic vs. Nuclear Membrane Permeabilization in a Whole Blood Sample using the Optimal Buffer Compositions. A) The CD45 vs. SS and FS vs. SS profiles of the samples after lysis. B) The degree of mitochondrial vs. nuclear membrane permeabilization in T cells. C) The degree of mitochondrial vs. nuclear membrane permeabilization in Monocytes. All of the detergent concentrations used in this experiment fully permeabilized the plasma membrane, while HSP60 and Lamin A/C indicate the degree of mitochondrial inner membrane and nuclear membrane permeabilization, respectively.

FIG. 6. Titration of Detergents In Order to Identify the Optimal Concentrations for Cytoplasmic vs. Nuclear Membrane Permeabilization of Granulocytes. The optimal permeabilization of the cytoplasm+nucleus can be seen with 0.0625% Digitonin+0.5% Tween 20. The optimal permeabilization of the cytoplasm alone, comparable to the whole-cell buffer, is 0.0625% Digitonin+0.25% TX-100. >0.5% Tween 20 alone will fully permeabilize the cytoplasm+mitochondria. In these graphs, the Tween 20 concentration is 2× the numbers indicated for the other detergents: it was titrated between 0.0625% and 1%. As in FIG. 5, HSP60 and Lamin A/C were used to indicate the degree of mitochondrial inner membrane and nuclear membrane permeabilization, respectively.

FIGS. 7A and 7B. Stimulation of Monocytes with 1 μg/mL LPS. A) Comparison of the scatter profiles between Buffer 1 and Buffer 2 lysis, as well as the gating workflow. B) Cytoplasmic vs. nuclear signaling in whole-blood monocytes. C) Cytoplasmic vs. nuclear signaling in T cells. LPS stimulated NF-κB and AKT signaling in monocytes, but did not stimulate T cells, as expected.

FIG. 8. Differential Signaling in Monocytes Induced by 1 μg/mL LPS vs. 100 ng/mL GM-CSF. A) Both LPS and GM-CSF induced CREB phosphorylation at S133, accumulating maximally in the nucleus by 10 min. B) LPS stimulation induced RelA phosphorylation maximally by 10 min in both the cytoplasm and nucleus, though predominantly in the nucleus. C) Both LPS and GM-CSF stimulated ERK phosphorylation primarily in the cytoplasm, maximally by 5 min for GM-CSF and 10 min for LPS.

FIG. 9. Stimulation of Intracellular Signaling in T Cells by CD3/CD28. CD3/CD28 induced CREB S133 phosphorylation maximally by 2.5 min, and RelA S536 phosphorylation maximally by 5 min, both primarily accumulating in the nucleus. The HDAC1 control is also shown to be predominantly in the nucleus.

FIG. 10. Analysis of STATS Nuclear Translocation in Tregs Following IL2 Stimulation. A) The gating of different CD25 subsets in the CD4 and CD8 T-cell populations. B) Analysis of the expression of FoxP3 in the different T-cell subsets gated in part A. In this chart, FoxP3 can be seen to be predominantly in the nucleus of the CD4+CD25hi population, which is expected since this is the Treg population. C) The nuclear translocation of STATS following IL2 stimulation in the different CD4 T-cell populations. IL2 stimulation induced maximal STATS translocation most rapidly in the Treg population, peaking by 2.5 min. The remaining CD4 T cells peaked by 10 min, with the CD25+population more strongly stimulated than the CD25low population. D) The nuclear translocation of STATS in the different CD8 T-cell populations. STATS translocation peaked by 10 min in the CD8+CD25+population, though was not induced in the CD8+CD25low population. All of these results are expected. The antibody used for STATS staining in this experiment was directed to the whole STATS protein, not to a phosphorylation site.

### DETAILED DESCRIPTION

### Overview

The present invention enables the quantitative determination of the subcellular localization of proteins within cells using standard labeling techniques in a variety of contexts, such as flow cytometry. This invention takes advantage of the differential ability of certain detergents to permeabilize the membranes of different subcellular organelles, each composed of different lipid compositions.

For example, the invention can be used directly on whole blood in a matter of hours, saving time and resources, thus increasing throughput and reducing costs. This invention is also very useful for analyzing rare cell populations within blood that may not be present in large enough quantities to effectively enable research with traditional techniques that first require their purification. Because purification of homogenous cell populations is not required, the present invention enables the analysis of cells in their endogenous state with much smaller sample quantities required compared to traditional techniques. Thus, the invention enables research with small sample volumes and can be used to study cell signaling in rare and precious samples (e.g., blood from pediatric patients), where the total volume of the sample is typically too low to conduct traditional research studies.

### Cell Sample

The cell sample in the methods of the present invention can be, for example, blood, bone marrow, spleen cells, lymph cells, bone marrow aspirates (or any cells obtained from bone marrow), urine (lavage), saliva, cerebral spinal fluid, urine, amniotic fluid, interstitial fluid, feces, mucus, tissue (e.g., tumor samples, disaggregated tissue, disaggregated solid tumor), or cell lines. In certain embodiments, the sample is a blood sample. In some embodiments, the blood sample is whole blood. The whole blood can be obtained from the subject using standard clinical procedures. In some embodiments, the sample is a subset of one or more cells, or cell-derived microvesicles or exosomes, from whole blood (e.g., erythrocytes, leukocytes, lymphocytes (e.g., T cells, B cells or NK cells), phagocytes, monocytes, macrophages, granulocytes, basophils, neutrophils, eosinophils, platelets, or any other cell, vesicle or exosome with one or more detectable markers). In some embodiments, the cells, or cell-derived microvesicles or exosomes, can be from a cell culture.

The subject can be a human (e.g., a patient suffering from cancer), or a commercially significant mammal, including, for example, a monkey, cow, or horse. Samples can also be obtained from household pets, including, for example, a dog or cat. In some embodiments, the subject is a laboratory animal used as an animal model of disease or for drug screening, for example, a mouse, a rat, a rabbit, or guinea pig. Samples may be primary or secondary tissues or cells that originated from such an organism.

### Target Analytes and Signal Transduction Pathway Activation

The target analyte of the present invention is typically a “signal-transduction pathway protein” or “activatable protein.” These terms are used to refer to a protein that has at least one isoform that corresponds to a specific form of the protein having a particular biological, biochemical, or physical property, e.g., an enzymatic activity, a modification (e.g., post-translational modification, such as phosphorylation), or a conformation. In a typical embodiment, the protein is activated through phosphorylation. As a result of activation, the protein is translocated to a different cellular compartment (e.g., from the cytoplasm to the nucleus).

The particular activatable protein targeted in the methods of the invention is not critical to the invention. Examples include member of the STAT family, such as STAT1, STAT2, STAT3, STAT4, STATS (STAT5A and STAT5B), and STATE. Extracellular binding of cytokines induce activation of receptor-associated Janus kinases, which phosphorylate a specific tyrosine residue within the STAT protein. The activated protein is then transported to the nucleus.

Examples of other activatable proteins include, but are not limited to, Histone deacetylase 1 (HDAC1), RELA (p65), cAMP response element-binding protein (CREB), Forkhead box P3 (FoxP3), ERK, S6, AKT, and p38.

An example of another signal transduction pathway includes the mitogen activated protein kinase (MAPK) pathway, which is a signal transduction pathway that affects gene regulation, and which controls cell proliferation and differentiation in response to extracellular signals. This pathway includes activatable proteins such as ERK1/2. This pathway can be activated by lipopolysaccharide (LPS), cytokines, such as interleukin-1 (IL-1) and tumor necrosis factor alpha (TNFα), CD40 Ligand, phorbol 12-myristate 13-acetate (PMA), and constitutively activated by proteins such as Mos, Raf, Ras, TPL2, and V12HaRas.

Another signal transduction pathway is the phosphatidylinositol-3-kinase (PI3K) pathway. The PI3K pathway mediates and regulates cellular apoptosis. The PI3K pathway also mediates cellular processes, including proliferation, growth, differentiation, motility, neovascularization, mitogenesis, transformation, viability, and senescence. The cellular factors that mediate the PI3K pathway include PI3K, AKT, and BAD.

Thus, in some embodiments the methods of the invention may include an activation step, which comprises the addition of an activator reagent to the cell sample. The activation reagent is adapted to trigger/activate at least one signal-transduction pathway within the cells. Suitable activator reagents include, for example, LPS, CD40L, PMA, or cytokines (e.g., IL-1, TNF, or GM-CSF). The activator reagent may also be one that constitutively activates the signal transduction pathway. Examples include proteins such as Mos, Raf, Ras, TPL2, and V12HaRas.

### Fixation and Permeabilization

The methods of the invention may include a fixation (or preservation) step that may include contacting the sample with a fixative in an amount sufficient to crosslink proteins, lipids, and nucleic acid molecules. Reagents for fixing cells in a sample are well known to those of skill in the art. Examples include aldehyde-based fixatives, such as formaldehyde, paraformaldehyde, and glutaraldehyde. Other fixatives include ethanol, methanol, osmium tetroxide, potassium dichromate, chromic acid, and potassium permanganate. In some embodiments a fixative may be heating, freezing, desiccation, a cross-linking agent, or an oxidizing agent.

As noted above, the methods of the invention include at least two permeabilization steps. The methods take advantage of the differential ability of detergents to permeabilize the membranes of different subcellular organelles, each composed of different lipid compositions. In the typical embodiment, one aliquot of cells from a cell sample is contacted with a first permeabilizing reagent that disrupts or lyses the cytoplasmic membrane (and possibly other membranes, such as the mitochondrial and ER membranes), but does not disrupt or lyse the nuclear membrane. A second aliquot of cells is contacted with a second permeabilizing reagent that disrupts or lyses the cytoplasmic membrane (and, the other membranes lysed by the first permeabilizing reagent), plus the nuclear membrane. In some embodiments, a third permeabilizing reagent may be used to lyse the cytoplasmic membrane and additional organelle membranes, with or without permeabilization of the nuclear membrane.

In a typical embodiment, each subsequent permeabilizing reagent will have a higher concentration of detergent than the previous permeabilizing reagent. Alternatively, the permeabilizing reagent may be composed of multiple detergents of different concentrations. In some embodiments the permeabilization steps may be carried out sequentially on the same sample.

The permeabilizing reagent (e.g., detergent) used to permeabilize the cells can be selected based on a variety of factors and can, for example, be an ionic or a non-ionic detergent. Suitable detergents are those that permeabilize cells and retain surface epitope integrity of the proteins being detected. Detergents are typically non-ionic detergents. Exemplary non-ionic detergents include Digitonin and ethyoxylated octylphenol (TRITON X-100®). Other useful permeabilizers (e.g., detergents) include Saponin, Polysorbate 20 (TWEEN® 20), Octylphenoxypoly(ethylene-oxy)ethanol (IGEPAL® CA-630) or Nonidet P-40 (NP-40), Brij-58, and linear alcohol alkoxylates, commercially available as PLURAFAC® A-38 (BASF Corp) or PLURAFAC® A-39 (BASF Corp). In some embodiments, ionic detergents, such as Sodium Dodecyl Sulfate (SDS), Sodium Deoxycholate, or N-Lauroylsarcosine, can be used.

### Binding Agents

A “binding agent” of the invention can be any molecule or complex of molecules capable of specifically binding to a target analyte (e.g., an activatable protein). A binding agent of the invention includes any molecule, e.g., proteins, small organic molecule, carbohydrates (including polysaccharides), oligonucleotides, polynucleotides, lipids, and the like. In some embodiments, the binding agent is an antibody or fragment thereof. Specific binding in the context of the present invention refers to a binding reaction which is determinative of the presence of a target protein in the presence of a heterogeneous population of proteins and other biological molecules. Thus, under designated assay conditions, the specified binding agents bind preferentially to a particular protein or isoform of the particular protein and do not bind in a significant amount to other proteins or other isoforms present in the sample.

When the binding agents are antibodies, they may be monoclonal or polyclonal antibodies. The term antibody as used herein refers to immunoglobulin molecules and immunologically active portions of immunoglobulin (Ig) molecules. Such antibodies include, but are not limited to, polyclonal, monoclonal, mono-specific polyclonal antibodies, antibody mimics, chimeric, single chain, Fab, Fab′ and F(ab′)₂ fragments, Fv, and an Fab expression library.

The binding agents of the invention may be labeled and are then referred to as “labeled binding agents”. A label is a molecule that can be directly (i.e., a primary label) or indirectly (i.e., a secondary label) detected. The label can be visualized and/or measured or otherwise identified so that its presence or absence can be detected by means of a detectable signal. Examples include fluorescent molecules, enzymes (e.g., horseradish peroxidase), particles (e.g., magnetic particles), metal tags, chromophores, phosphors, chemiluminescers, specific binding molecules (e.g., biotin and streptavidin, digoxin and antidigoxin), and the like.

In a typical embodiment, the label is a fluorescent label, which is any molecule that can be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 Oregon green, green fluorescent protein (GFP), blue fluorescent protein (BFP), enhanced yellow fluorescent protein (EYFP), and luciferase. Additional labels for use in the present invention include: Alexa-Fluor dyes (such as: Alexa Fluor 350, Alexa Fluor 430, Alexa Fluor 488, Alexa Fluor 546, Alexa Fluor 568, Alexa Fluor 594, Alexa Fluor 633, Alexa Fluor 660, and Alexa Fluor 680), conjugated polymer-based dyes, dendrimer-based dyes, quantum dots, polymer dots, and phycoerythrin (PE).

In certain embodiments, multiple fluorescent labels are employed with the capture molecules of the present invention. In some embodiments, at least two fluorescent labels may be used which are members of a fluorescence resonance energy transfer (FRET) pair. FRET pairs (donor/acceptor) useful in the invention include, but are not limited to, PE-Cy5, PE-Cy5.5, PE-Cy7, APC-Cy5, APC-Cy7, APC-AF700, APC-AF750, EDANS/fluorescein, IAEDANS/fluorescein, fluorescein/tetramethylrhodamine, fluorescein/LC Red 640, fluorescein/Cy5, fluorescein/Cy5.5, and fluorescein/LC Red 705.

Conjugation of the label to the capture molecule can be performed using standard procedures well known in the art. For example, conventional methods are available to bind the label moiety covalently to proteins or polypeptides. Coupling agents, such as dialdehydes, carbodiimides, dimaleimides, bis-imidates, bis-diazotized benzidine, and the like, can be used to label antibodies with the above described fluorescent, chemiluminescent, and enzymatic labels.

Although the methods of the invention do not require that the binding agent be specific for the activated (e.g., phosphorylated) forms of the activatable proteins, such binding agents may be used in the claimed methods. Antibodies, many of which are commercially available, have been produced which specifically bind to the phosphorylated isoform of a protein but do not specifically bind to a non-phosphorylated isoform of a protein. Exemplary antibodies for p-ERK include Phospho-p44/42 MAPK (ERK1/2) clones E10 or D13.14.4E, which are commercially available from Cell Signaling Technology.

Other examples of labeled binding agents include, without limitation, the following antibodies: Mouse anti-Stat5 (pY694)-PE (BD Biosciences Pharmingen San Jose Calif.), Mouse Phospho-p44/42 MAPK (ERK1/2) (Thr202/Tyr204) (E10) Alexa Fluor 647, Phospho-p38 MAPK (T180/Y182) Alexa Fluor 488, Phospho-Statl (Tyr701) (58D6) Alexa Fluor 488, Phospho-Stat3 (Tyr705) (3E2) Alexa Fluor 488 (Cell Signaling Technology Inc., Danvers, Mass.), Phospho-AKT (Ser473) (A88915), Phospho-p44/42 MAPK (ERK1/2) (Thr202/Tyr204) (A88921), Phospho-Stat3 (Tyr705) (A88925), Phospho-p38 MAPK (Thr180/Tyr182) (A88933), Phospho-S6 Ribosomal Protein (Ser235/236) (A88936), Phospho-Statl (Tyr701) (A88941), and Phospho-SAPK/JNK (Thr183/Tyr185) (A88944, Beckman Coulter Inc. (BCI), Brea, Calif.).

In some embodiments, a binding agent that specifically binds a cellular surface antigen or surface marker can be used. Examples of surface markers include transmembrane proteins (e.g., receptors), membrane associated proteins (e.g., receptors), membrane components, cell wall components, and other components of a cell accessible by an agent at least partially exterior to the cell. In some embodiments, a surface marker is a marker or identifier of a type or subtype of cell (e.g., type of lymphocyte or monocyte). In some embodiments, a surface marker is selected from the group consisting of: CD1, CD2, CD3, CD4, CD5, CD6, CD8, CD10, CD11a, CD14, CD15, CD16, CD19, CD20, CD24, CD25, CD26, CD27, CD28, CD29, CD30, CD31, CD32, CD33, CD34, CD38, CD40, CD45, CD45RA, CD45RO, CD49a-f, CD53, CD54, CD56, CD61, CD62L, CD64, CD69, CD70, CD80, CD86, CD91, CD95, CD114, CD117, CD120a, CD120b, CD127, CD134, CD138, CD152, CD153, CD154, CD161, CD181, CD182, CD183, CD184, CD185, CD186, CD191, CD192, CD193, CD194, CD195, CD196, CD197, CD198, CD199, CD252, CD257, CD268, CD273, CD274, CD275, CD278, CD279, CD281, CD282, CD283, CD284, CD286, CD288, CD289, CD290, CD326, and CD357.

### Measurement Systems

Measurement systems utilizing a binding agent and a label to quantify bound molecules in cells are well known. Examples of such systems include flow cytometers, scanning cytometers, imaging cytometers, imaging flow cytometers, fluorescence microscopes, confocal fluorescent microscopes, and mass cytometers.

In some embodiments, flow cytometry may be used to detect fluorescence. A number of devices suitable for this use are available and known to those skilled in the art. Examples include Beckman Coulter Navios, Gallios, Aquios, and CytoFLEX flow cytometers. In some embodiments, if metal-tagged antibodies are utilized, the cells may be analyzed using mass cytometry.

### Kits

The reagents useful in the methods of the invention can also be produced in the form of kits. Such kits are a packaged combination comprising, for example, the basic elements of: (a) a first permeabilizing reagent that permeabilizes the cytoplasmic membrane of cells but does not permeabilize the nuclear membrane of cells; and (b) a second permeabilizing reagent that permeabilizes both the cytoplasmic membrane and the nuclear membrane of the cells. The kit may also comprise (c) a labeled binding agent which specifically binds a control (e.g., organelle-specific or cytoskeletal proteins) or an activatable protein (e.g., a phosphorylated form, anunphosphorylated form, or both), (d) a fixative, and (e) instructions on how to perform the method using these reagents. In some embodiments, a wash buffer may also be included.

An exemplary kit is composed of two separate buffers for whole-blood mononuclear cells (i.e., lymphocytes+monocytes): 1) The first buffer is to permeabilize the cytoplasm, including the ER, the endosomal system, and the outer mitochondrial membrane, while 2) the second buffer is to permeabilize everything permeabilized by the first buffer, plus the nucleus (and, in some embodiments, the inner mitochondrial matrix). Buffer 1 (Cytoplasm) may be composed of: 1-100 mM MES pH 4.5-6.5, 0-274 mM NaCl, 0-5.4 mM KCl, and 0.01-0.15% Digitonin. The optimal detergent concentration for Buffer 1 is between 0.001 and 0.25% Digitonin, where the cytoplasm is lysed but the nucleus is not. Buffer 2 (Whole Cell) may be composed of: 1-100 mM MES pH 4.5-6.5, 0-274 mM NaCl, 0-5.4 mM KCl, and >0.01% Digitonin or >0.0125% Triton X-100. The optimal detergent concentration for Buffer 2 depends on the sample type, with the upper bound limited by the loss of surface markers and the disintegration of cells that occurs around 2% for both.

For both Buffer 1 and Buffer 2, the salt concentrations may be anywhere between 0 and 4× of the given 1× concentration in physiological saline: i.e., 0-274 mM NaCl+0-5.2 mM KCl. With some detergents, differences in salt concentrations may affect the efficiency of targeting of specific cellular membranes. The fixative can be, for example, composed of: 8-10% Paraformaldehyde in 1×PBS (10-20 mM NaH2PO4 pH 7.4, 137 mM NaCl, and 2.7 mM KCl), providing a final fixative concentration of 4-5%. Buffers 1 and 2 will also work with a final fixative concentration anywhere between 1 and 10%, though protein modifications with cell signaling will be less preserved at lower concentrations, and the lysis of RBCs is more efficient at concentrations >4%. The salt concentration in the fixative will work between 0 and 2× of the given 1× concentration, though the light scatter profiles for the WBCs may be affected a little bit at the lower concentrations, and the effectiveness of the detergents decreases as the concentration approaches 2×.

### EXAMPLES

The following examples are offered to illustrate, but not to limit, the claimed invention.

The purpose of this invention is to enable the quantitative determination of the subcellular localization of proteins within cells by flow cytometry, as well as other cytometric techniques. This system functions by taking advantage of the differential ability of certain detergents to permeabilize the membranes of different subcellular organelles, each composed of different lipid compositions.

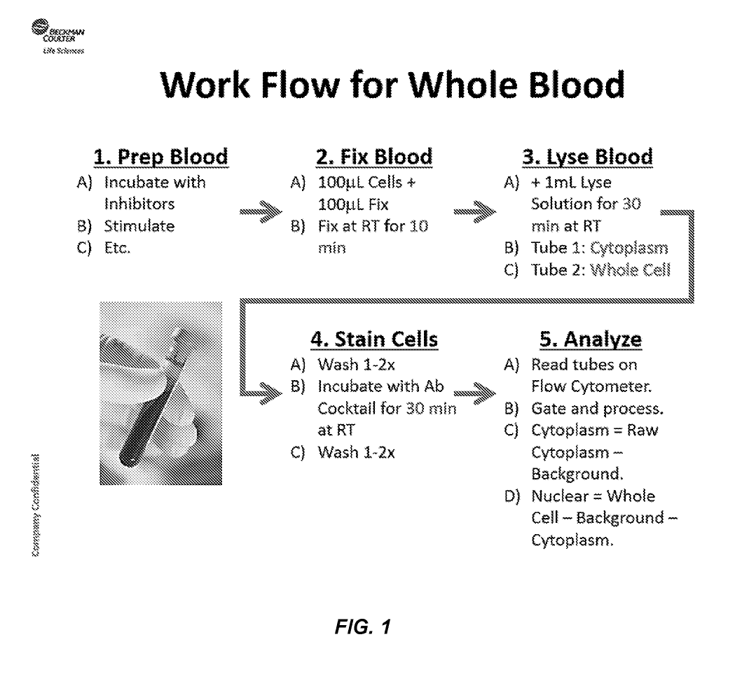

The protocol for processing whole blood samples is as follows (see FIG. 1 for a workflow): 1) The sample is first mixed 1:1 with fixative, vortexed, and then incubated for 10 minutes. An extra control tube is included for each buffer, which is stained with all antibodies except for the specific signaling or target antibodies being tested in order to subtract the background signal. The background control may also be labeled with isotype-control antibodies for more precise determination of non-specific binding, especially in cells that have characteristically high non-specific binding, such as neutrophils. For indirect antibody labeling, omitting the primary antibody, but still utilizing the secondary antibody, is a common method for determining the degree of non-specific background signal attributable to the secondary antibody, which is typically higher than the direct conjugates of target antibodies. 2) During the fixation period, the samples are split into 2 separate fractions, one for cytoplasmic lysis and the other for whole-cell lysis. Alternatively, 2 separate tubes may be set up in advance for each sample, assuming that they are both treated the same. 3) After fixation, the sample in each tube is mixed 1:5 with Buffer 1 or Buffer 2, respectively (e.g., 2004 of sample (including fixative)+1 mL of lysis buffer); the Background control is also lysed with each buffer, though it may only be necessary to lyse with one of the two if both buffers are composed of the same detergent (even if at different concentrations). The tubes are then vortexed and incubated for 15-30 minutes at RT. 4) After lysis, the samples are washed 2× with PBS or standard wash buffer (e.g., PBS+1% BSA), and then stained for 30 minutes with the desired antibody cocktail. 5) If unconjugated primary antibodies are used, the samples may be washed and stained with secondary antibodies with/without immunophenotyping antibodies. The immunophenotyping antibodies may require another wash and then a blocking step in order to prevent non-specific binding to any secondary antibodies that target their host species. 6) Once stained, the samples are again washed 2× with PBS or wash buffer, resuspended in PBS+0.5% PFA, and read on a flow cytometer.

After data acquisition, the samples are gated, compensated, and analyzed as standard flow cytometry samples. In order to determine cytoplasmic vs. nuclear localization, the resulting data are further processed as follows: 1) For the Cytoplasm: The target signals from the Background Control for Buffer 1 are subtracted from the raw Cytoplasmic data. 2) For the Nucleus: The target signals from the Background Control for Buffer 2 are first subtracted from the Whole Cell data, and then the processed Cytoplasm data are further subtracted from this result. For example, if staining for the subcellular distribution of FoxP3, where the Background MFIs for the Cytoplasm and Whole Cell are 1.5 and the raw FoxP3 signal is 3.5 and 31.5 for the Cytoplasm and Whole Cell, respectively, then the Cytoplasmic MFI would be calculated to be 2 (i.e., 3.5 (raw)−1.5 (background)=2) and the Nuclear MFI would be calculated to be 28 (i.e., 31.5 (raw)−1.5 (background)−2 (Cytoplasm)=28). If the same detergent is used in both Buffer 1 and 2, then it may be possible to simplify the data processing by subtracting the raw Cytoplasmic data from the Whole Cell to obtain the Nuclear data, without intermittently subtracting the background. This is also demonstrated in the previous example (i.e., the Nuclear MFI would simply be calculated as: 31.5 (raw)−3.5 (raw Cytoplasm)=28). A small percentage of some select proteins may be present within the inner mitochondrial matrix, but this would be expected to have a very small effect on the nuclear localization data for such proteins, if any (very small fraction of the signal), and would not be expected to change the activation-dependent translocation signals for most proteins, including transcription factors, due to the requirement for protein denaturation in order to cross both the outer and inner mitochondrial membranes, the necessity to refold within the inner mitochondrial matrix in order to perform a function, and the fact that the mitochondria is simply a different system that doesn't utilize most cellular proteins: it is a remnant bacteria.

For Peripheral Blood Mononuclear Cells (PBMCs), cell lines, and other purified cells, Buffer 1 and Buffer 2 have different compositions than for whole blood; however, the protocol is otherwise the same. Most cell lines perform similar to PBMCs. In addition, whole-blood granulocytes may require a different buffer combination to appropriately permeabilize their cytoplasmic vs. nuclear compartments. Specifically, Buffer 1 composed with 0.0625% Digitonin+0.25% TX-100 will lyse the plasma membrane without lysing the mitochondria or nucleus, while Buffer 2 composed with 0.0625% Digitonin+>0.125% Tween 20 will lyse the plasma membrane (comparable to Buffer 1) and will also fully lyse the nucleus; >0.5% Tween 20 by itself will lyse the plasma membrane and completely lyse the mitochondria without lysing the nucleus, while low concentrations of TX-100 or Digitonin alone will lyse the mitochondria or nucleus, respectively, though not at higher concentrations.

### Example 1

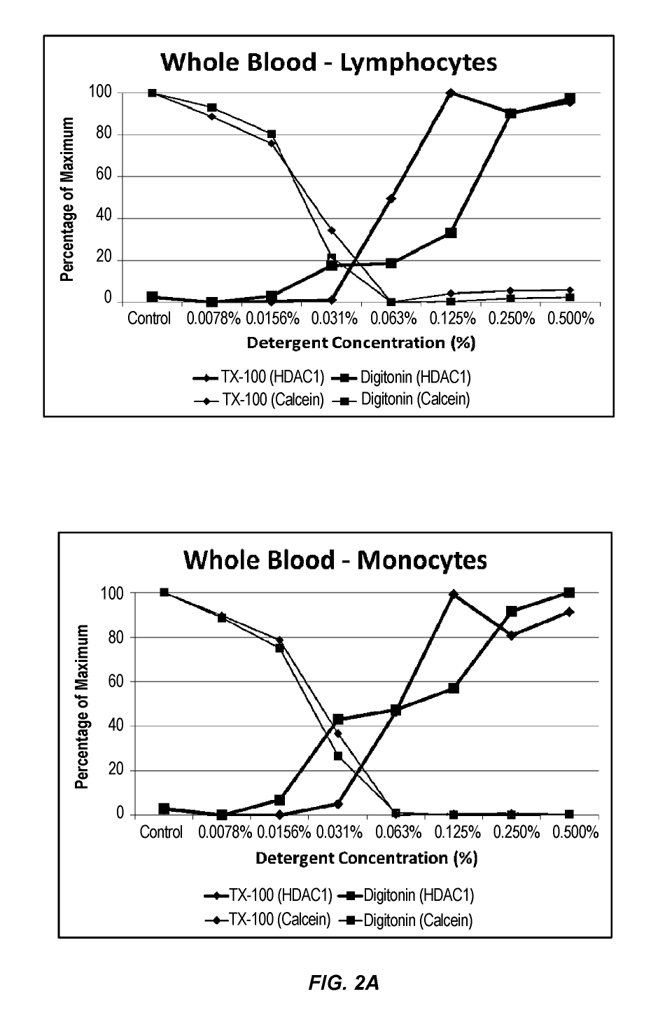

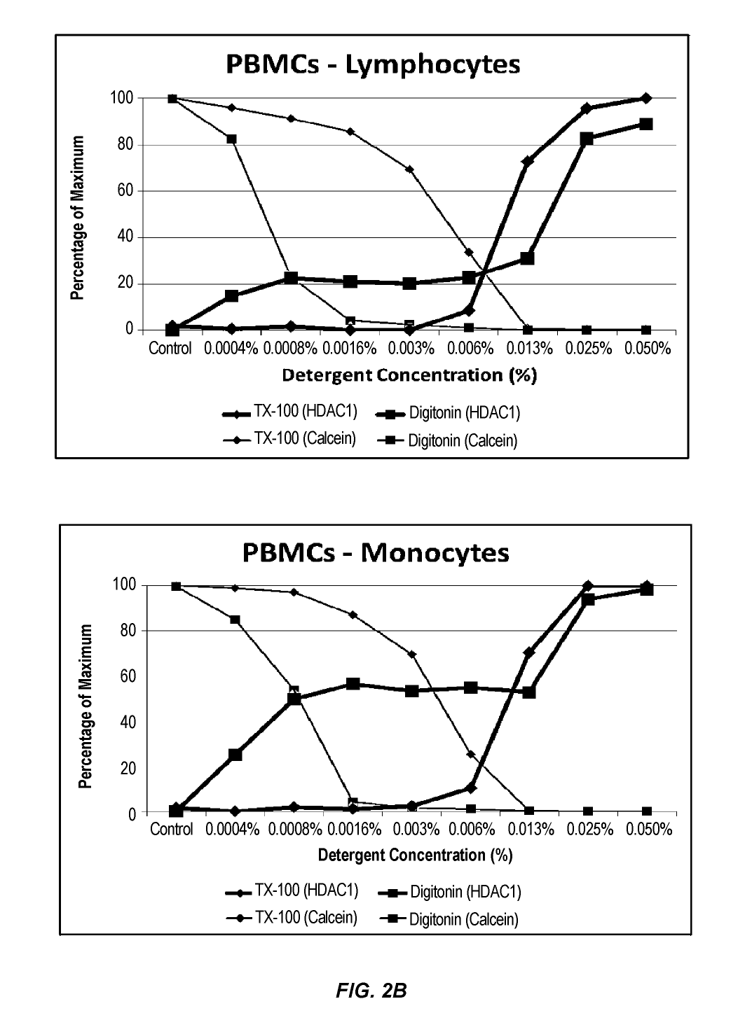

FIG. 2 is a comparison of the efficiency of cytoplasmic vs. nuclear permeabilization of T cells and monocytes by different concentrations of Digitonin or TX-100. FIG. 2A is a titration performed on whole blood, while FIG. 2B is a titration performed on PBMCs. In both cases, the samples were first preloaded for 1 hour with 1 μM CytoCalcein Violet (AAT Bioquest, Inc) in a CO2-regulated 37° C. incubator. After 1 hour, the samples were fixed for 10 min with 4% PFA, and then incubated for 30 min at RT with the different concentrations of detergents diluted in diH2O, at a 1:5 ratio with the sample mixture. The samples were then washed and stained with anti-HDAC1-FITC (Abcam, Plc), washed again, and finally read on a Gallios flow cytometer (BCI). In this figure, cytoplasmic lysis is indicated by the loss of CytoCalcein Violet signal as it is released from the cell once the plasma membrane is permeabilized, while nuclear lysis is indicated by the increased HDAC1 signal as the nuclear membrane is permeabilized. In the case of Digitonin lysis, there is a ledge of HDAC1 staining once the plasma membrane is lysed and prior to full nuclear lysis; this is indicative of lysis of the endoplasmic reticulum, which also contains a repository of HDAC1. In whole blood, there is a working range for Digitonin between roughly 0.015% and 0.125%, where the plasma membrane is lysed, but the nucleus is not. For PBMCs, this range is roughly between 0.001% and 0.0125%. TX-100 does not provide this working range, and begins lysing the nucleus almost immediately after a sufficient concentration is reached for plasma membrane permeabilization. Complete lysis of the cells is achieved at a concentration of either 0.25% Digitonin or 0.125% TX-100 with whole blood, and either 0.025% Digitonin or 0.025% TX-100 with PBMCs.

### Example 2

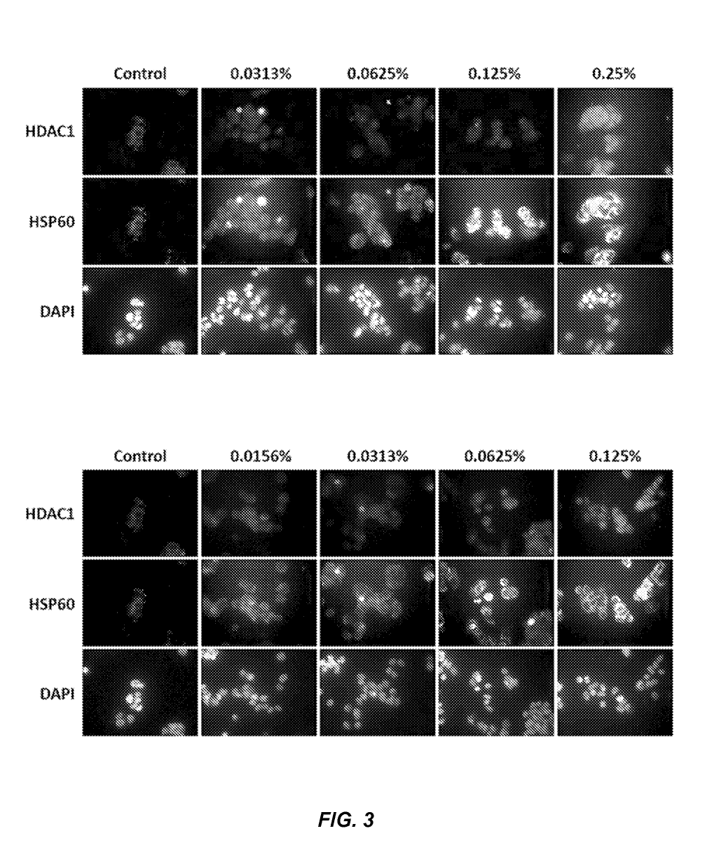

FIG. 3 depicts a titration of Digitonin or TX-100 with MCF-7 cells, a breast cancer cell line. FIG. 3A is a titration of Digitonin, while FIG. 3B is a titration of TX-100. In both cases, the cells were cultured for 24 hours prior to experimentation in 8-well glass microscope slides (Nunc). On the day of experimentation, the cells were first fixed for 10 min with 4% PFA and then incubated for 30 minutes at RT with the different concentrations of detergents diluted in 1×PBS. The samples were then washed and labeled for 1 hour at RT with mouse-anti-human-HSP60 and rabbit-anti-human-HDAC1 antibodies (Santa Cruz Biotechnologies). After 1 hour, the samples were washed again and labeled for 30 min at RT with chicken-anti-mouse-AF488 and chicken-anti-rabbit-AF647 antibodies (Molecular Probes). Finally, the samples were washed, coverslipped with Vectashield mounting medium containing DAPI (Vector Laboratories), and images were captured using a Zeiss Axioskop 2 Plus fluorescence microscope together with a 63× oil-immersion lens. In FIG. 3A, Digitonin can be seen to permeabilize the cytoplasm beginning around 0.031%, as indicated by HSP60 staining in the cytoplasm and mitochondria; while it began to fully permeabilize the nucleus around 0.25%, as indicated by the increased HDAC1 staining within the nucleus. In FIG. 3B, TX-100 can be seen to permeabilize the cytoplasm beginning around 0.016%, and then the nucleus beginning around 0.125%.

### Example 3

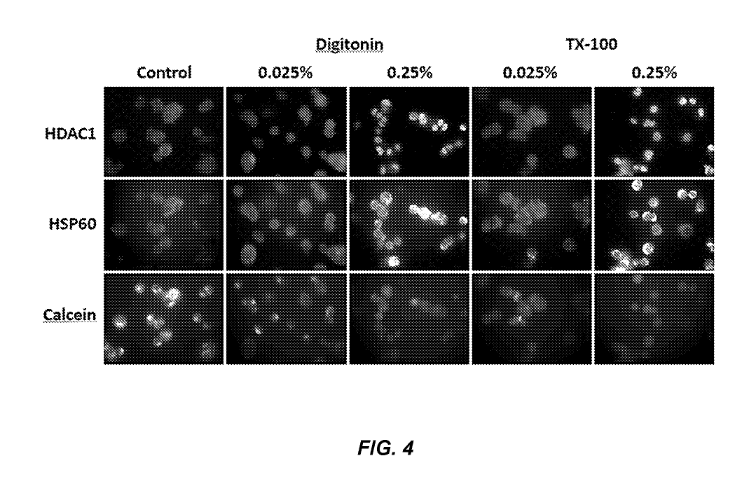

FIG. 4 was a modification of the protocol for staining MCF-7 cells in order to more clearly demonstrate the permeabilization of the plasma membrane, and to eliminate the possibility that lower levels of apparent HSP60 staining may have been due to non-specific binding of the secondary antibody. In this experiment, after the cells had been plated for 24 hours, they were preloaded for 1 hour with 1 μM CytoCalcein Violet and then processed as indicated in FIG. 3. When the samples were ready for cover-slipping, mounting medium without DAPI was used. Images were captured on a Zeiss Axioskop 2 Plus microscope together with a 63× oil-immersion lens. In this figure, MCF-7 cells that were not permeabilized can be seen to be loaded with CytoCalcein Violet, and this staining is lost once the plasma membrane is permeabilized. Closer inspection of the subcellular localization of the CytoCalcein Violet indicates that it is loaded within the endosomal system, as would be expected for the time frame utilized when loading the cells. In turn, the loss of CytoCalcein Violet staining upon cytoplasmic permeabilization indicates that the endosomal system is also permeabilized, which is expected because the endosomal membranes pinch off from the plasma membrane. In this experiment, 0.025% of both Digitonin and TX-100 can be seen to permeabilize the plasma membrane, while 0.25% of both can be seen to permeabilize the whole cell. This modified protocol was used for subsequent testing of the performance of the different detergents with whole blood and PBMCs by flow cytometry, including for the results in FIG. 2.

### Example 4

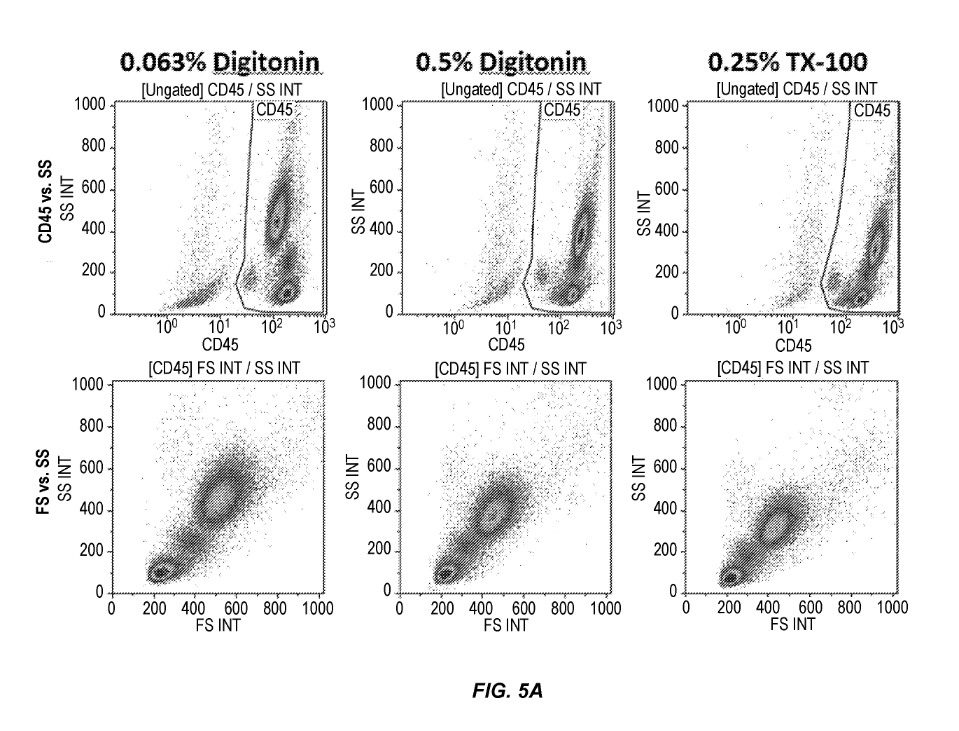

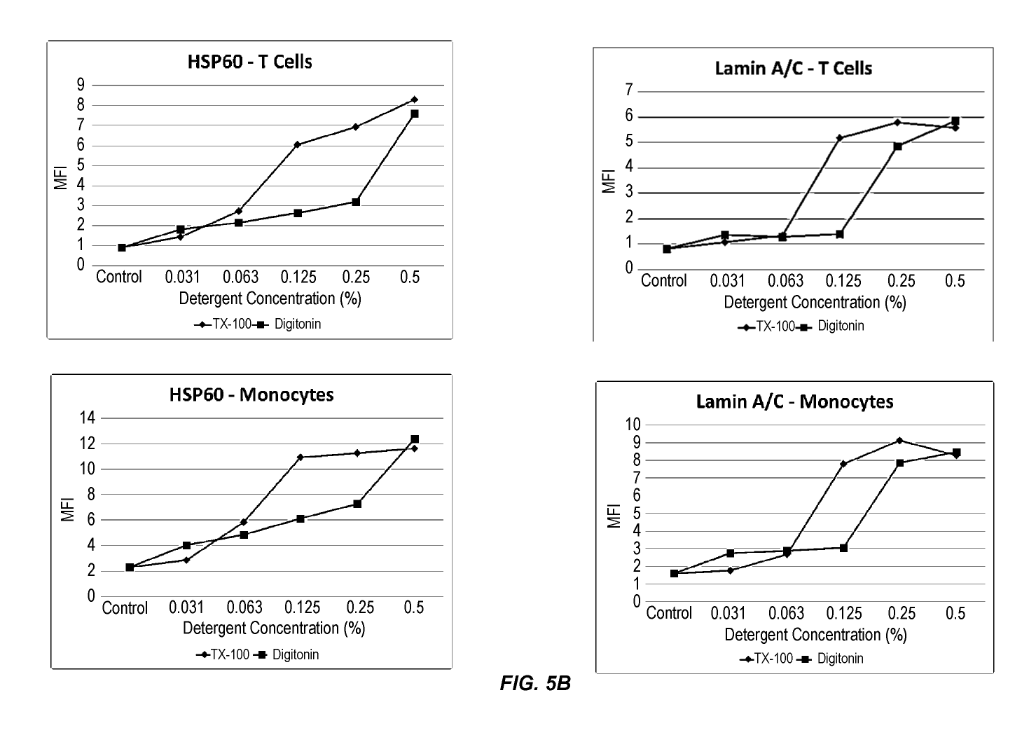

FIG. 5 indicates the optimal lysis parameters for whole blood, including modified buffer conditions in order to improve RBC lysis. While the detergents were found to perform well to differentially permeabilize cellular membranes when diluted in diH₂O, the diH₂O was found to be inconsistent in its effectiveness with lysing RBCs at very low detergent concentrations, especially if the fixation time extended by more than a couple minutes beyond protocol. In order to improve RBC lysis, the solution was ultimately buffered with MES at a pH between 4.5-6.5 (such as a pH of 5.5), which also allowed the salt concentration to be increased to physiological levels. This further improved the scatter profiles of the WBCs, decreased the time required for complete lysis to approximately 15 minutes, and improved the RBC lysis efficiency to the point that the buffers still work well if the fixation time is extended well beyond protocol (>20 min). At the same time, the fixative concentration was increased to 5% due to improved performance. In FIG. 5A, the scatter profiles for the optimal lysis parameters can be seen, where 0.0625% Digitonin is optimal for cytoplasmic membrane permeabilization, and either 0.5% Digitonin or 0.25% TX-100 are optimal for whole-cell membrane permeabilization. In the CD45 vs. SS plots, the RBCs can be seen to be completely lysed, while the FS vs. SS plots demonstrate the retained WBC scatter profiles at the different concentrations. FIG. 5B demonstrates the effectiveness of mitochondrial (HSP60) and nuclear (Lamin A/C) membrane permeabilization in T cells, while FIG. 5C demonstrates the same in Monocytes. In both cases, the permeabilization profiles can be seen to be consistent with the defined optimal detergent concentrations.

### Example 5

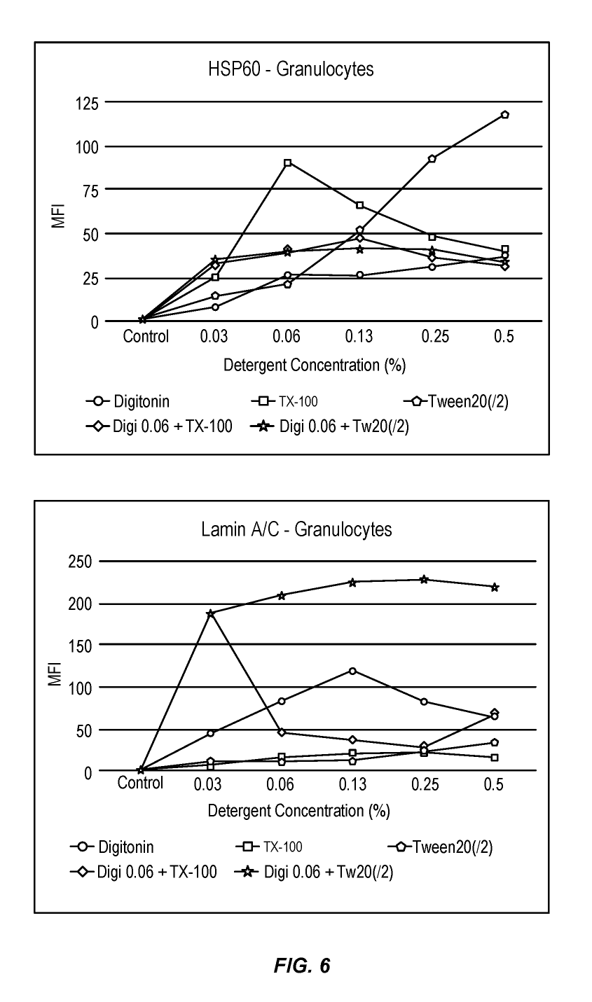

FIG. 6 demonstrates the optimal detergent combinations for the differential permeabilization of granulocytes. In some cases, using the defined buffers for the Subcellular Localization Kit may not effectively and reproducibly permeabilize granulocytes as they do mononuclear cells, and can be better targeted with a different detergent combination. As can be seen in FIG. 6, the cytoplasmic+nuclear membranes of granulocytes are optimally permeabilized by 0.0625% Digitonin+0.5% Tween 20 (the Tween 20 concentrations in the graph are 2× the concentrations indicated for the other detergents), while the cytoplasmic membrane alone is most comparably permeabilized by 0.0625% Digitonin+0.25% TX-100. Tween 20 at a concentration >0.5% can be seen to permeabilized the cytoplasmic+mitochondrial membranes without permeabilizing the nuclear membrane, while Digitonin and TX-100 alone at lower concentrations will permeabilize the nuclear or mitochondrial membranes, respectively. Ultimately, differential permeabilization of granulocytes can be more complex than for mononuclear cells depending on the target organelles.

### Example 6

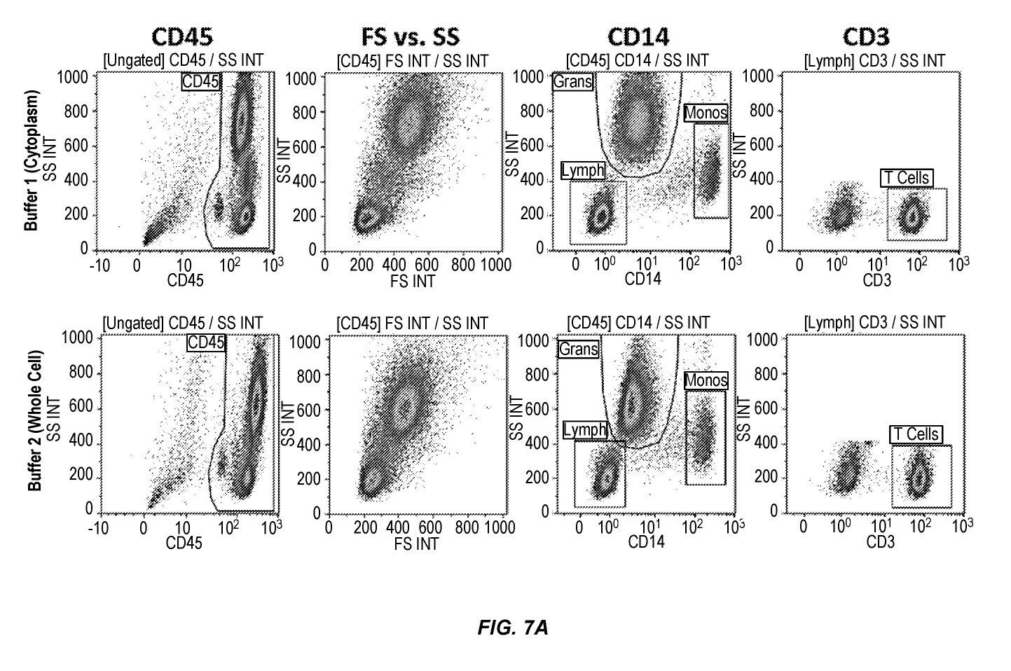

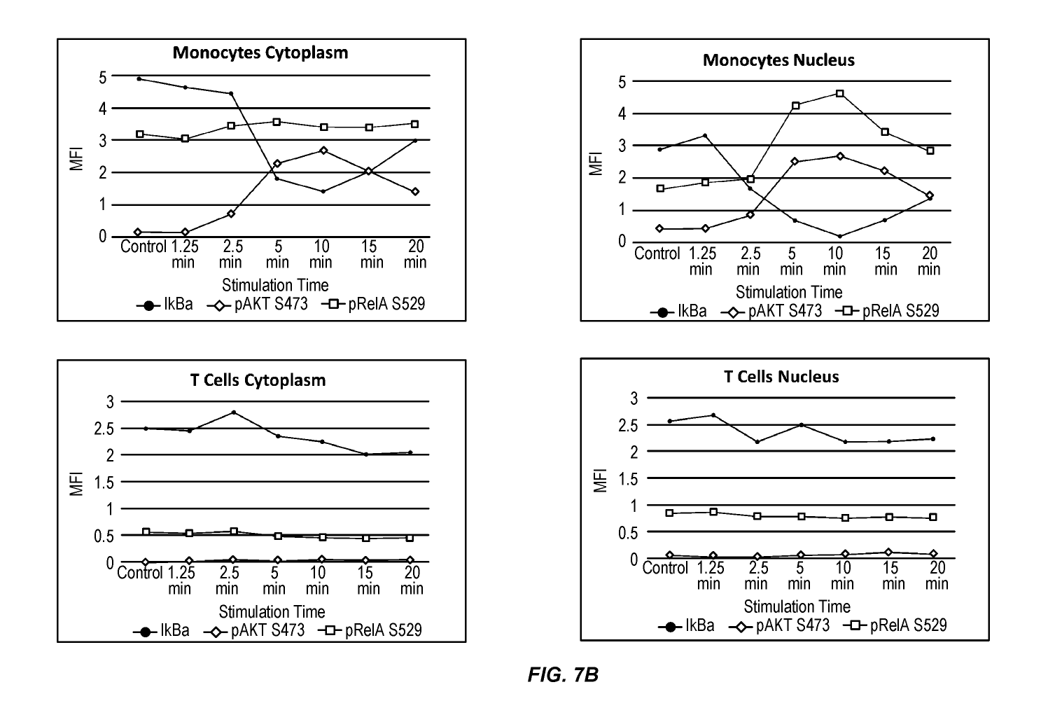

FIG. 7 demonstrates the analysis of cell signaling in LPS-stimulated monocytes. Whole blood was stimulated with 1 μg/mL LPS for the indicated times. The samples were then fixed with 5% PFA and processed with the buffer compositions for the Subcellular Localization Kit, using 0.0625% Digitonin for Buffer 1 and 0.5% Digitonin for Buffer 2. In FIG. 7A, the scatter profiles for Buffer 1 vs. Buffer 2 lysis can be seen, together with the gating workflow for the different WBC populations. In FIG. 7B, IκBa can be seen to be degraded in both the cytoplasm and nucleus, while AKT is phosphorylated at S473 in both the cytoplasm and nucleus, and RelA phosphorylated at S529 builds up within the nucleus, all maximally by 10 min. In contrast, FIG. 7C shows a lack of any signaling induced in T cells. These results are expected, as LPS stimulates the TLR4 receptors on monocytes, using CD14 as a co-receptor, which are not present on T cells.

### Example 7

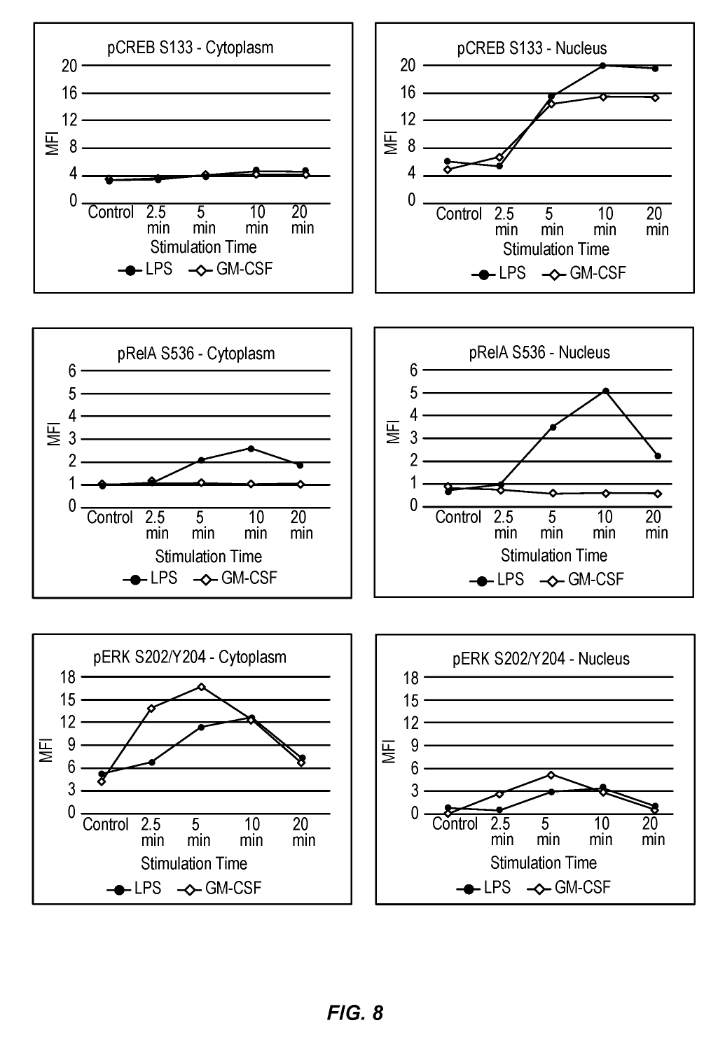

FIG. 8 demonstrates another stimulation of monocytes with either 1 μg/mL LPS or 100 ng/mL GM-CSF. In this experiment, the cells were fixed with 4% PFA and processed with 0.05% Digitonin for Buffer 1 and 0.5% Digitonin for Buffer 2, both diluted in diH₂O. In FIG. 8A, the induction of CREB phosphorylation at S133 is shown, building to a maximum at 10 min in the nucleus for both stimulations. In FIG. 8B, the induction of RelA phosphorylation at 5536 can be seen to peak around 10 min in the nucleus following LPS stimulation, and to also accumulate in the cytoplasm to a lower degree. GM-CSF did not stimulate RelA phosphorylation at S536. In FIG. 8C, ERK phosphorylation at S202/T204 can be seen to be induced by both LPS and GM-CSF primarily in the cytoplasm, and to a smaller degree in the nucleus. This phosphorylation peaked by 5 min for GM-CSF and 10 min for LPS.

### Example 8

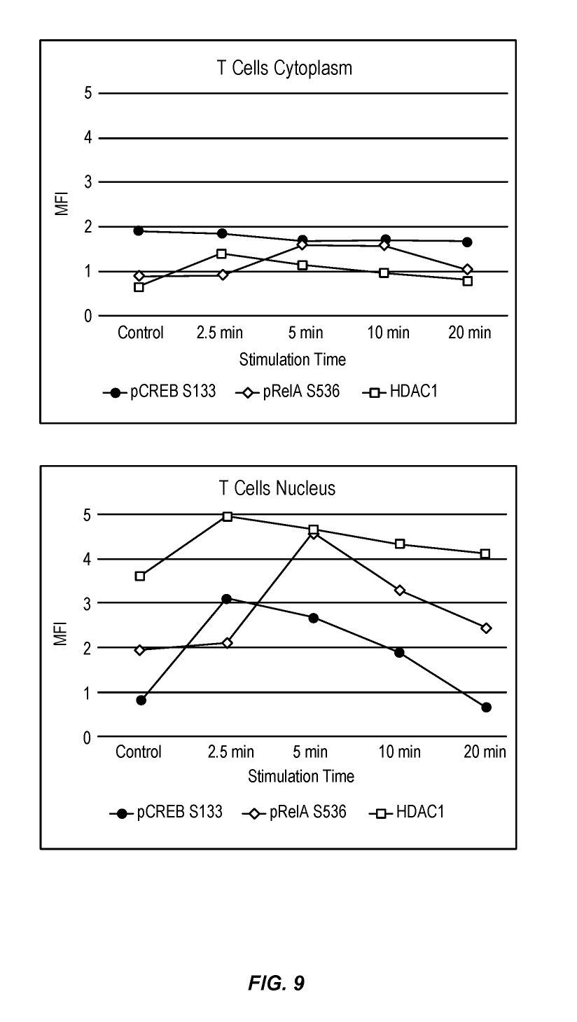

FIG. 9 shows the stimulation of T cells with 0.25 μg/mL CD3 (OKT3)+2.5 μg/mL CD28 (CD28.2) (BD Biosciences)+10 μg/mL goat-anti-mouse crosslinker (Jackson ImmunoResearch). In this experiment, the samples were fixed with 4% PFA and processed with 0.05% Digitonin for Buffer 1 and 0.5% Digitonin for Buffer 2, both diluted in diH₂O. Following CD3/CD28 stimulation, pCREB 5133 built up within the nucleus maximally by 2.5 min, while pRelA S536 built up within the nucleus and to a smaller degree in the cytoplasm by 5 min. HDAC1 staining is also shown to be predominantly located within the nucleus, as expected.

### Example 9

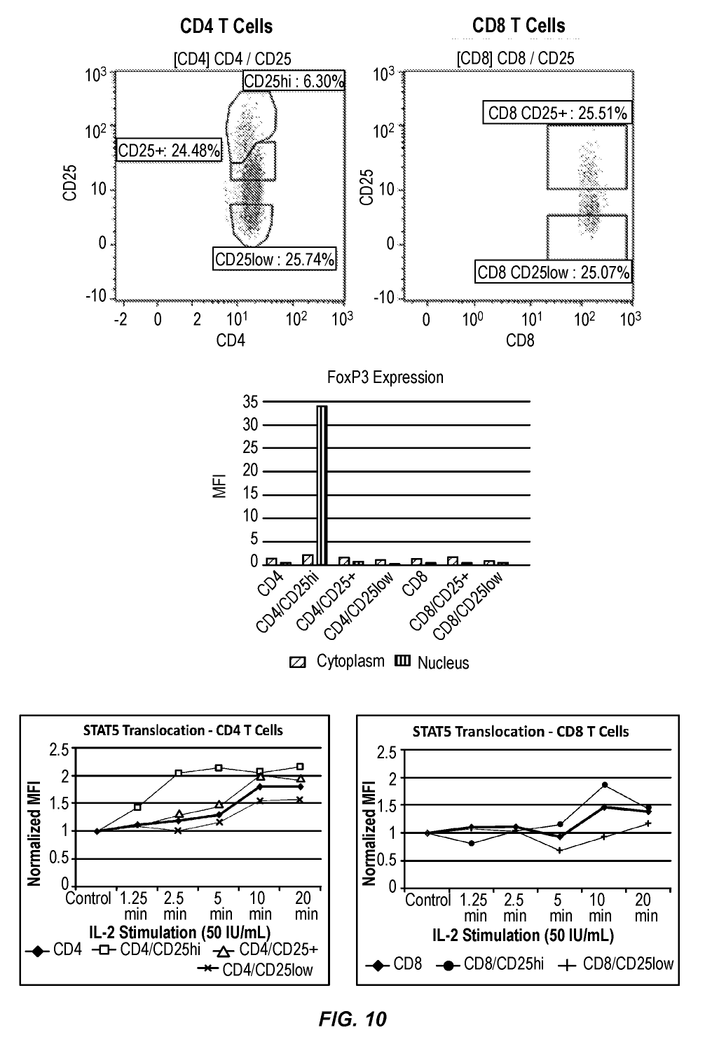

FIG. 10 depicts the preferential activation of STATS nuclear translocation in Tregs following stimulation with 501U/mL of IL2. In this experiment, the samples were fixed with 4% PFA and processed with 0.05% Digitonin for Buffer 1 and 0.5% Digitonin for Buffer 2, both diluted in diH₂O. FIG. 10A shows the gating of the CD25hi, CD25+, and CD25low populations of CD4 and CD8 T cells. FIG. 10B shows the cytoplasmic vs. nuclear localization of FoxP3+stained with anti-FoxP3-AF647 (BCI). In this graph, FoxP3 can be seen to be predominantly localized within the nucleus of the CD4+CD25hi cells, which is expected since this is the Treg population, defined by FoxP3 expression in the nucleus. FIG. 10C shows the nuclear translocation of the whole STATS protein detected using anti-STATS-FITC (Abcam). In this figure, STATS can be seen to translocate most rapidly into the nucleus of the Treg population, peaking near 2.5 min, while its translocation was induced more slowly in CD4+CD25+cells, peaking at 10 min. STATS translocation was also induced maximally by 10 min in CD8+CD25+cells, though to a lesser degree. The ability to detect the nuclear translocation of the whole STATS protein, without requiring the detection of STATS phosphorylation, is a demonstration of the power of this technique to work around the limitations of existing techniques that can only detect differences in protein modifications: if an epitope for an antibody to a whole protein is not exposed, there is always another antibody to a different epitope available; this is not the case for specific protein-modification sites.

### Alternative Approaches:

The methods of the invention rely on different detergents or detergent concentrations in order to gently lyse the cytoplasm plus as many cytoplasmic components as possible in one tube, and the whole cell including the nucleus in the other tube. For this reason, a variety of detergents will work to accomplish this task. Some are as follows, with reference to their performance with whole blood:

### Cytoplasm:

Saponin (Quillaja bark): >0.03% will permeabilize the cytoplasmic membrane of Lymphocytes and Monocytes without permeabilizing any apparent subcellular organelles. For granulocytes, it will also permeabilize the nuclear membrane at lower concentrations. This may be a viable alternative to Digitonin (another member of the Saponin family) for cytoplasmic membrane permeabilization, though higher concentrations will not permeabilize the nuclear membrane. Higher concentrations of Saponin may also be used for Buffer 1 to match the osmolarity of the 2 buffers if necessary. However, Saponin produces a higher background signal than Digitonin.

Tween 20: There is a range between roughly 0.0625% and 0.25% where the plasma membrane will be completely permeabilized and the nucleus is untouched for Lymphocytes and Monocytes. The cytoplasmic+mitochondrial membranes will be completely permeabilized in granulocytes as the concentration increases, which is indicated above. Tween 20 also greatly alters the surface tension of the solution, and will coat the test tubes making them very slick. This offers one benefit in that it helps to completely rid the tube of buffer with little effort when decanting between washes, but it produces a great disadvantage in that it is hard to properly resuspend the sample with small volumes of antibody cocktail for staining.

TX-100: There is a tight range right at 0.0313% and possibly up to 0.0625% where the cytoplasmic membrane will be permeabilized without affecting the nucleus for Lymphocytes and Monocytes. However, this may be too narrow for consistent performance with different donors.

NP-40 (Igepal CA-630) performs equivalently to TX-100.

Titrating low levels of ionic detergents, such as Sodium Dodecyl Sulfate, Sodium Deoxycholate, or N-Lauroylsarcosine, together with low levels of non-ionic detergents to permeabilize the cytoplasmic membrane, will completely permeabilize the cytoplasmic+mitochondrial membranes, but will inhibit nuclear membrane permeabilization at lower concentrations. At concentrations greater than roughly 0.125%-0.25%, the ionic detergents will begin to denature proteins before reaching concentrations high enough to permeabilize the nucleus. Once the concentrations are reached that will permeabilize the nucleus, the scatter profiles begin to degrade and typically 1 more titration step will completely disintegrate the sample. This may be useful for compartmentalizing the mitochondria with Buffer 1 at lower concentrations, but differences in the levels of protein denaturation between Buffers 1 and 2 would ultimately complicate the reliability of the assay. Moreover, different proteins are denatured at different concentrations of ionic detergents, so it may be impossible to predefine the expected performance for the entire proteome.

### Whole Cell:

Digitonin at 0.0625%+TX-100 at 0.125-0.25% will completely permeabilize cells better than either Digitonin or TX-100 alone. However, it degrades the sample scatter profiles more than either detergent alone, and the degree of degradation of sample quality is not always consistent.

Using Digitonin at 0.0625% to permeabilize the cytoplasmic membrane will allow combination with lower levels of other detergents, such as CHAPS and Sodium Deoxycholate, to also permeabilize the nucleus. However, the performance of CHAPS decreases at lower pHs, and Sodium Deoxycholate immediately precipitates out of solution at pHs lower than ˜7.0 regardless of the concentration. Therefore, these may be useful for PBMCs or cell lines where a reduced pH is not necessary, but not for whole blood.

Saponin may be interchangeable with Digitonin for combining with other detergents to accomplish whole-cell permeabilization. However, as previously mentioned, the background will typically be a little higher than that of other detergents.

NP-40 is interchangeable with TX-100 for whole-cell permeabilization.

Ionic detergents such as Sodium Dodecyl Sulfate and N-Lauroylsarcosine will completely permeabilize the cells when used alone at higher concentrations. However, they also denature proteins, which may make achieving equivalency between Buffers 1 and 2 difficult, as previously mentioned.

The pH of the buffers affects their performance with RBC and platelet lysis. The optimal pH range for the buffers is between 4.5 and 6.5. A pH below 4.5 begins to greatly damage the scatter profiles and increase platelet granularity, while a pH above 6.5 will result in decreased RBC lysis efficiency after 10-15 min of fixation. The optimal pH is between 5 and 6. This pH range can be accomplished using a variety of buffers other than MES, including citrate, phosphate, and others that have useful ranges that at least partially overlap with the pH 4.5-6.5 range.

The protocol may also be modified to reduce the quantity of detergent required as follows: 1) First, fix the samples and lyse the RBCs with the MES-buffered saline alone (i.e., 1-100 mM MES, pH4.5-6.5, 0-274 mM NaCl, and 0-5.4 mM KCl), without any added detergents. 2) Then, wash the sample, concentrate the WBCs by centrifugation, and decant the buffer and debris. 3) Finally, permeabilize the enriched WBCs in a smaller volume of detergent, such as 50-200 uL of 0.01-0.15% Digitonin either in the MES-buffered saline or even PBS for the cytoplasm or whole cell, respectively. With the smaller volume, the staining antibodies may be included together with the permeabilization buffer, resulting in a roughly equivalent processing time. The rate of RBC lysis by the MES-buffered saline alone may actually be increased by either increasing the concentration of MES or other buffer, switching buffers (e.g., citrate is more rapid than MES at an equivalent concentration), changing the salt concentration, or possibly supplementing the buffer with low concentrations of Saponin or Digitonin, as long as these modifications do not affect the specificity of the cytoplasmic vs. whole cell permeabilization in Step 3. If the cytoplasmic membrane is permeabilized during the RBC-lysis step, such as with Saponin, then the second permeabilization step may be modified to target specific subcellular organelles, without any additional detergent required for the Cytoplasmic tube, and with the possibility of targeting specific membranes with lower concentrations of detergents than would typically be possible if they have to first overcome the plasma membrane. However, the use of multiple detergents and/or multiple detergent-lysis steps tends to degrade sample quality, epitopes, and scatter profiles pretty greatly, regardless of the detergent combinations.

The protocol may also be performed sequentially so that the signals can be seen and compared within individual cells. This method can be performed as follows: 1) Fix the sample and permeabilize the cytoplasmic membrane+RBCs with 0.0625% Digitonin. 2) Wash the sample. 3) Stain the cytoplasmic analytes with the first antibody or other marker. 4) Wash the sample again, and preferably crosslink the antibodies or other markers from step 3 prior to proceeding. 5) Permeabilize the nucleus either with or without the remaining antibodies or markers to stain the remaining analytes. 6) Optional: If not stained together with nuclear permeabilization, stain the remaining analytes in this step. 7) Wash and resuspend in PBS/0.5% PFA. 8) Analyze the sample with a flow cytometer or microscope. The second permeabilization step could either require the same 0.0625% Digitonin concentration as the first step if lysed using 1 mL volume, or >0.0625% Digitonin if utilizing a smaller 50-200 μL volume as indicated above. In order to discriminate the cytoplasmic from nuclear signal in this case, the 2 antibodies would necessitate different labels, and thus the signals for the 2 compartments could not be directly compared quantitatively (i.e., they would be qualitative between compartments). However, the differences within compartments would be quantitative. The primary disadvantage of this protocol is the time required to perform the sequential permeabilization, staining and washing steps being roughly double that of the standard protocol.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of quantifying an analyte within a sample of cells, the method comprising:
treating a first aliquot of the cells with a first permeabilizing reagent that permeabilizes the cytoplasmic membrane but does not permeabilize the nuclear membrane;
treating a second aliquot of the cells with a second permeabilizing reagent that permeabilizes both the cytoplasmic membrane and the nuclear membrane;
washing the first and the second aliquots
staining the first aliquot and the second aliquot with a labeled reagent capable of specifically binding to the analyte;
measuring a first signal from the labeled reagent in a cell of the first aliquot and a second signal from the labeled reagent in a cell of the second aliquot; and
comparing the first signal to the second signal to determine the distribution of the analyte.

2. The method of claim 1, wherein the step of measuring includes measuring on a cell-by-cell basis the first signal from a plurality of cells of the first aliquot and the second signal from a plurality of cells of the second aliquot.

3. The method of claim 1, wherein the step of measuring on a cell-by-cell basis includes measuring in a cytometer.

4. The method of claim 1, further comprising treating a third aliquot of the cells with a third permeabilizing reagent that permeabilizes the cytoplasmic membrane and an organelle membrane.

5. The method of claim 1, wherein the first reagent includes between 0.001 and 0.25% Digitonin.

6. The method of claim 5, wherein the first permeabilizing reagent includes about 0.01-0.15% Digitonin.

7. The method of claim 5, wherein the first permeabilizing reagent includes about 1-100 mM MES at pH 4.5-6.5, 0-274 mM NaCl and 0-5.2 mM KCl.

8. The method of claim 7, wherein the first permeabilizing reagent includes about 137 mM NaCl, and about 2.7 mM KCl.

9. The method of claim 1, wherein the second permeabilizing reagent includes one of >0.01% Digitonin or >0.0125% TX-100.

10. The method of claim 9, wherein the second permeabilizing reagent includes one of about 0.025-0.5% Digitonin or about 0.0125-0.25% Triton X-100.

11. The method of claim 9, wherein the second permeabilizing reagent includes about 1-100 mM MES at pH4.5-6.5, 0-274 mM NaCl and 0-5.2 mM KCl.

12. The method of claim 1, wherein the step of treating the first aliquot of the cells includes fixing the cells with a fixative.

13. The method of claim 12, wherein the fixative includes about 1-10% paraformaldehyde.

14. The method of claim 1, wherein the cells include mononuclear cells.

15. The method of claim 1, wherein the analyte is an activatable protein, a protein constitutively present in one compartment or another, a protein differentially expressed or activated in diseased or aberrant samples, DNA, RNA, peptides, or sugars.

16. The method of claim 15, wherein the activatable protein is a transcription factor, a kinase, a phosphatase, a DNA- or RNA-binding or modifying protein, a nuclear import or export receptor, a regulator of apoptosis or cell survival, a ubiquitin or ubiquitin-like protein, or a ubiquitin or ubiquitin-like modifying enzyme.

17. The method of claim 15, where the protein constitutively present in one compartment or another is a structural protein, organelle-specific marker, proteasome, transmembrane protein, surface receptor, nuclear pore protein, protein/peptide translocase, protein folding chaperone, signaling scaffold, or ion channels.

18. The method of claim 15, where the analyte may also be the DNA, chromosomes, oligonucleotides, polynucleotides, RNA, mRNA, tRNA, rRNA, microRNA, peptides, polypeptides, proteins, lipids, ions, monosaccharides, oligosaccharides, polysaccharides, lipoproteins, glycoproteins, glycolipids, or fragments thereof.

19. The method of claim 1, wherein the cells include granulocytes and the first permeabilizing reagent includes one of a mixture of about 0.01-0.15% Digitonin and about 0.0125-0.25% TX-100, and the second reagent contains a mixture of about 0.01-0.15% Digitonin and >0.0125% Tween 20 or >0.05% Tween 20.

20. The method of claim 1, wherein the step of staining the first aliquot and the second aliquot includes staining the first aliquot and the second aliquot with a labeled reagent capable of specifically binding to a surface marker of the cells.

21. A kit for quantifying an analyte within a sample of cells, the kit comprising:
a first permeabilizing reagent that permeabilizes the cytoplasmic membrane of the cells but does not permeabilize the nuclear membrane of the cells; and
a second permeabilizing reagent that permeabilizes both the cytoplasmic membrane and the nuclear membrane of the cells.

22. The kit of claim 21, wherein the first permeabilizing reagent includes one of about 0.01-0.15% Digitonin or a mixture of about 0.01-0.15% Digitonin and about 0.0125-0.25% TX-100.

23. The kit of claim 21, wherein the second permeabilizing reagent includes one of about 0.025-0.5% Digitonin, 0.0125-0.25% TX-100, 0.01-0.15% Digitonin and >0.0125% Tween 20, or >0.05% Tween 20.

24. The kit of claim 21, further comprising a fixative.
